(19) United States
(12) Patent Application Publication
(10) Pub. No.: US 2025/0129527 A1
(43) Pub. Date: Apr. 24, 2025

(54) WOVEN FABRIC BIOELECTRONIC DEVICE

(71) Applicants: CZ BIOHUB SF, LLC, San Francisco, CA (US); THE REGENTS OF THE UNIVERSITY OF CALIFORNIA, Oakland, CA (US)

(72) Inventors: Michel M. Maharbiz, El Cerrito, CA (US); Wentian Mi, Berkeley, CA (US); Harmanvir Singh Ghuman, Alameda, CA (US); Karunesh Ganguly, Oakland, CA (US)

(21) Appl. No.: 18/836,459

(22) PCT Filed: Feb. 7, 2023
(86) PCT No.: PCT/US23/12461

§ 371 Date: Aug. 7, 2024

Related U.S. Application Data
(60) Provisional application No. 63/307,974, filed on Feb. 8, 2022.

(51) Int. Cl. D03D 15/67; A61L 31/00; A61L 31/16; A61M 31/00; A61N 1/05; A61N 5/06

(52) U.S. Cl. CPC D03D 15/67; A61L 31/005; A61L 31/16; A61M 31/00; A61N 1/0536; A61N 5/0603; A61L 2300/414; A61M 2205/04; A61M 2205/051; A61M 2205/054; A61M 2210/0693; A61N 2005/063; A61N 2005/0663; D10B 2401/18; D10B 2401/20; D10B 2509/022

(57) ABSTRACT

The following relates generally to an implantable device (e.g., a device that is implantable to a human or animal subject). In some embodiments. the implantable device comprises a woven portion comprising two or more pluralities of threads woven together. Each of the pluralities of threads may be of a different thread type. Examples of types of threads include electrical wire. permeable tubing. optical fiber. polymer fiber, surface functionalized fiber. absorbable suture, and/or fiber comprising material configured for sensing. In some embodiments, the implantable device may be used to repair a lesion (e.g., a tumor).

### CROSS-REFERENCE TO RELATED APPLICATIONS

This application claims priority to and the benefit of U.S. Application No. 63/307,974, filed on Feb. 8, 2022, entitled “WOVEN FABRIC BIOELECTRONIC DEVICE,” the entire disclosure of which is hereby expressly incorporated by reference herein.

### STATEMENT OF GOVERNMENT INTEREST

This invention was made with government support under 5101RX001640 awarded by United States Department of Veterans Affairs (VA). The government has certain rights in the invention.

### BACKGROUND

It is sometimes useful to implant a device into a subject. For example, an implantable device may be inserted into the brain of a human subject to deliver electrical stimulation to the brain. However, current implantable devices have several drawbacks. For instance, current implantable devices are rigid (e.g., inflexible), difficult to fabricate, and/or serve only one purpose (e.g., not able to perform any useful functions besides deliver electrical stimulation).

The systems and methods disclosed herein provide solutions to these problems and others.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one aspect, an implantable device may be provided. The implantable device may comprise: (1) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and (2) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing.

In another aspect, a method of repairing a lesion may be provided. The method may comprise: (1) growing a first group of cells on an implantable device, the first group of cells comprising induced pluripotent stem cells (iPSCs), neural stem cells (NSCs), or neurons, and the implantable device comprising: (i) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and (ii) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing; and (2) inserting the implantable device into a subject.

In yet another aspect, a method of repairing a lesion may be provided. The method may comprise: (1) inserting a first group of cells into the subject, the first group of cells comprising induced pluripotent stem cells (iPSCs), neural stem cells (NSCs), or neurons; and (2) inserting an implantable device into a subject, the implantable device comprising: (i) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and (ii) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing.

### BRIEF DESCRIPTION OF THE DRAWINGS

FIG. 1 illustrates an example system for using an implantable device, according to an embodiment.

FIG. 2 illustrates an example part of an example woven portion of the implantable device.

FIGS. 3A, 3B and 3C show the warp threads woven together with the weft threads according to various configurations.

FIG. 4 illustrates an example woven portion including electrical wire threads and optical fiber threads.

FIG. 5 illustrates an example of an implantable device, including permeable tubing.

FIG. 6 illustrates an example method of repairing a lesion, including growing a first group of cells on the implantable device prior to insertion of the implantable device into the subject.

FIG. 7 illustrates an example method of repairing a lesion, including inserting a first group of cells into the subject prior to inserting the implantable device into the subject.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The present embodiments relate to, inter alia, an implantable device.

By way of background, current implantable devices have several drawbacks. In particular, current implantable devices are rigid (e.g., inflexible), and/or serve only one purpose (e.g., not able to perform any useful functions besides deliver electrical stimulation). The systems and methods disclosed herein provide elegant solutions to these problems and others.

More specifically, in some embodiments, the disclosed implantable device comprises threads woven together. In this regard, building the device of woven threads achieves many advantages. For example, by using different types of threads in the device, a multifunctional device is achieved. For example, when some of the threads are electrical wires and others are optical fibers, the implantable device may deliver both electrical stimulation and optical stimulation.

In the disclosed implantable device, any types of threads may be used. For example, the types of threads may include any of: electrical wire, permeable tubing, optical fiber, polymer fiber, surface functionalized fiber, absorbable suture, and/or fiber comprising material configured for sensing.

In another advantage, weaving the threads together achieves a flexible yet structurally stable device. To even further this advantage, some embodiments include structural threads (e.g., for stability), and functional threads (e.g., to provide additional functions). Examples of structural threads include polymer fiber, and absorbable suture. Examples of functional threads include electrical wire, permeable tubing, optical fiber, surface functionalized fiber, and fiber comprising material configured for sensing. It should be understood that some embodiments comprise only two types of threads woven together. However, other embodiments comprise more than two types of threads woven together, and any number of types of threads may be used.

In some embodiments, the implantable device may be used to repair a lesion (e.g., a tumor) of a subject. To this end, in some implementations, prior to insertion of the implantable device into the subject, a group of cells (e.g., induced pluripotent stem cells (iPSCs), neural stem cells (NSCs), and/or neurons) are grown on the implantable device. Subsequently, the implantable device is inserted into the subject (e.g., into a lesion of the subject).

In other implementations that repair a lesion of the subject, a group of cells (e.g., iPSCs, NSCs, and/or neurons) are first inserted into the subject (e.g., into the lesion of the subject), and subsequently the implantable device is inserted into the subject (e.g., into or near the lesion).

Once inserted into the subject, the implantable device may then be advantageously used to monitor progress of the subject (e.g., healing of the lesion), deliver growth factors, deliver drugs, provide electrical stimulation, provide optical stimulation, and so forth, as will be discussed further herein.

### Example System

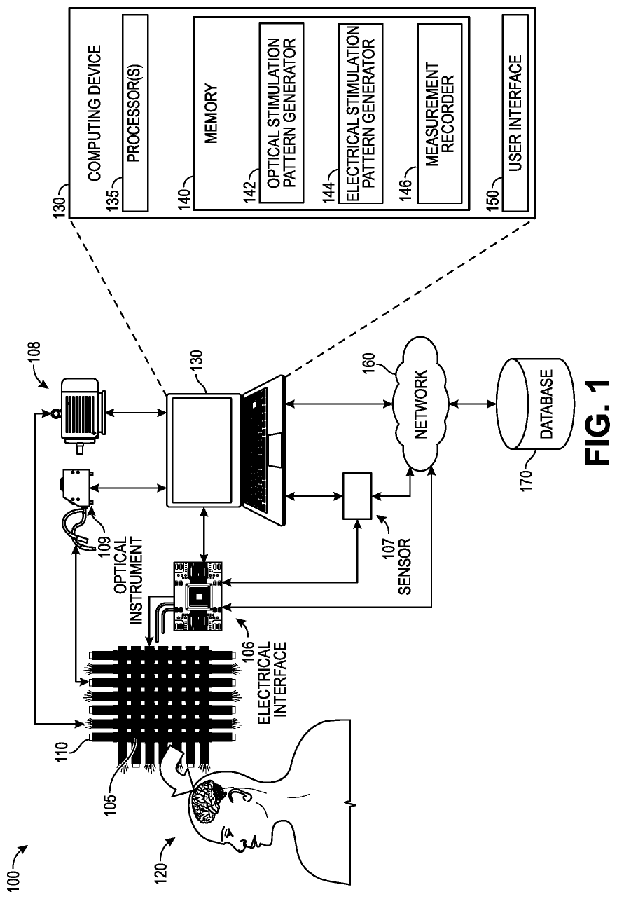

FIG. 1 illustrates an example system 100 for using an implantable device 110, according to an embodiment. In the example system 100, the implantable device 110 may be implanted in a brain or any other part of the subject 120. It should be understood that although the example of FIG. 1 illustrates the subject 120 as a human, the subject 120 does not necessarily have to be human; for example, the subject 120 may be any other kind of animal, such as another kind of mammal, a reptile, a bird, etc.

The implantable device 110 may include a woven portion 105, and/or an electrical interface 106. The electrical interface 106 may be configured to communicate (e.g., directly; or via a network 160, which may be a wired or wireless network, such as the internet) with a computing device 130.

The computing device 130 may include one or more processors 135 such as one or more microprocessors, controllers, and/or any other suitable type of processor. The computing device 130 may further include a memory 140 (e.g., volatile memory, non-volatile memory) accessible by the one or more processors 135 (e.g., via a memory controller). Additionally, the computing device 130 may include a user interface 150.

The one or more processors 135 may interact with the memory 140 to obtain, for example, computer-readable instructions stored in the memory 140. Additionally or alternatively, computer-readable instructions may be stored on one or more removable media (e.g., a compact disc, a digital versatile disc, removable flash memory, etc.) that may be coupled to the computing device 130 to provide access to the computer-readable instructions stored thereon. In particular, the computer-readable instructions stored on the memory 140 may include instructions for executing various applications, such as optical stimulation pattern generator 142, electrical stimulation pattern generator 144, and/or measurement recorder 146. The computing device 130 may further be in communication with a database 170 for storing/retrieving any kind of information. For example, the database 170 may be the database of a hospital that stores patient information, such as information of subject 120. Furthermore, it should be understood that although the example of FIG. 1 illustrates only one database 170, any number of databases may be in communication with the computing device 130.

The computing device 130 may comprise any suitable device. For example, the computing device 130 may comprise server(s), personal computer(s), a smartphone, a tablet, a phablet, etc. In some embodiments, the computing device 130 stores information received from the electrical interface 106, and controls the implantable device 110 (e.g., through the electrical interface 106).

For example, the computing device 130 may control the implantable device to deliver optical stimulation (e.g., through optical fibers) and/or electrical stimulation (e.g., through electrical wires) to the subject 120. For instance, the computing device 130 may control the optical instrument 109 (e.g., a fiber optic illuminator, a laser generator, etc.) to deliver optical stimulation

Furthermore, the computing device 130 may also control the pump 108 to deliver a chemical or biological substance to the subject 120 (e.g., though permeable tube(s) of the woven portion 105). The computing device 130 may be in communication with the pump 108 in any suitable way (e.g., an electrical connection, such as an Ethernet connection; a wireless connection, such as Bluetooth; etc.). In addition, although not shown in the example of FIG. 1, the pump 108 may be connected to the network 160; and thus, the computing device 130 may be in communication with the pump 108 through the network 160 as well. In addition, although the example of FIG. 1 illustrates only one pump 108, any number of pumps may be used.

The example of FIG. 1 also includes sensor 107, which may be connected to the implantable device 105 through the electrical interface 106. The sensor 107 may sense a chemical or biological substance of the subject 120 (e.g., though permeable tube(s) of the woven portion 105). The computing device 130 may be in communication with the sensor 107 in any suitable way (e.g., an electrical connection, such as an Ethernet connection; a wireless connection, such as Bluetooth; etc.). In addition, the sensor 107 may be connected to the network 160; and thus, the computing device 130 may be in communication with the sensor 107 through the network 160 as well. In addition, although the example of FIG. 1 illustrates only one sensor 107, any number of sensors may be used.

In addition, any data of the subject 120 may be recorded. For example, if a lesion is being repaired, data of the subject's 120 progress may be recorded. For example, any measurements taken via the sensor 107 may be recorded. In other examples, electrical measurements and/or optical measurements may be recorded. In some implementations, the data is recorded using the measurement recorder 146. The data may be stored in the memory 140, the database 170, etc. In some embodiments, the data is stored in the database 170, and the database 170 is a hospital database connected to a hospital network, thus conveniently allowing access to the recorded data at other locations.

### Example Woven Portions

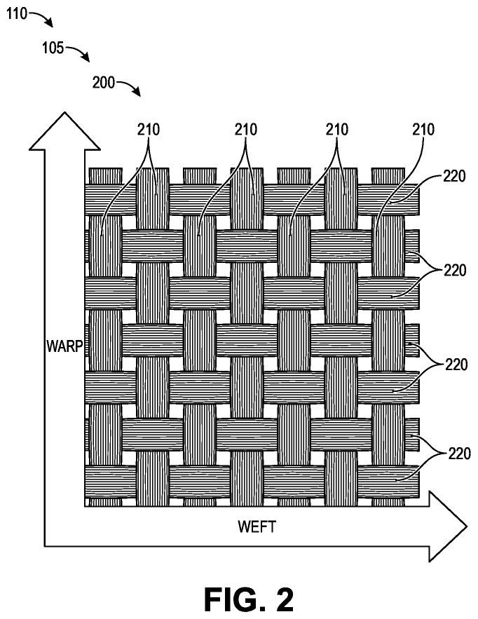

FIG. 2 illustrates an example part 200 of an example woven portion 105 of the implantable device 110. The illustrated example includes warp threads 210 and weft threads 220. In the illustrated example, the warp threads 210 are orientated in a first direction (e.g., a warp direction), and the weft threads 220 are orientated in a second direction (e.g. a weft direction) with the first direction being perpendicular to the second direction.

It should be understood that the warp threads 210 and/or weft threads 220 may comprise any of the types of threads (or combinations of types of threads) discussed herein. Moreover, individual threads of the warp threads 210 may all be the same type of thread, or different types of threads. Similarly, individual threads of the weft threads 220 may all be the same type of thread, or different types of threads. Examples of types of threads include electrical wire, permeable tubing, optical fiber, polymer fiber, surface functionalized fiber, absorbable suture, or fiber comprising material configured for sensing. In some embodiments, each of the threads may have a diameter of between 1-100 μM.

To illustrate, in one example, the warp threads 210 may comprise a first plurality of threads with the first plurality of threads having a thread type of electrical wire; and the weft threads 220 may comprise a second plurality of threads with the second plurality of threads having a thread type of polymer fiber. In another example, the weft threads 220 may comprise a first plurality of threads with the first plurality of threads having a thread type of permeable tubing; and the warp threads 210 may comprise a second plurality of threads with the second plurality of threads having a thread type of absorbable suture.

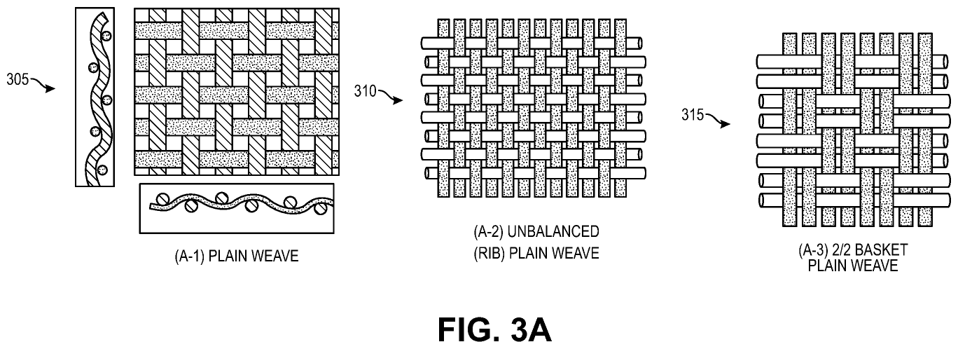

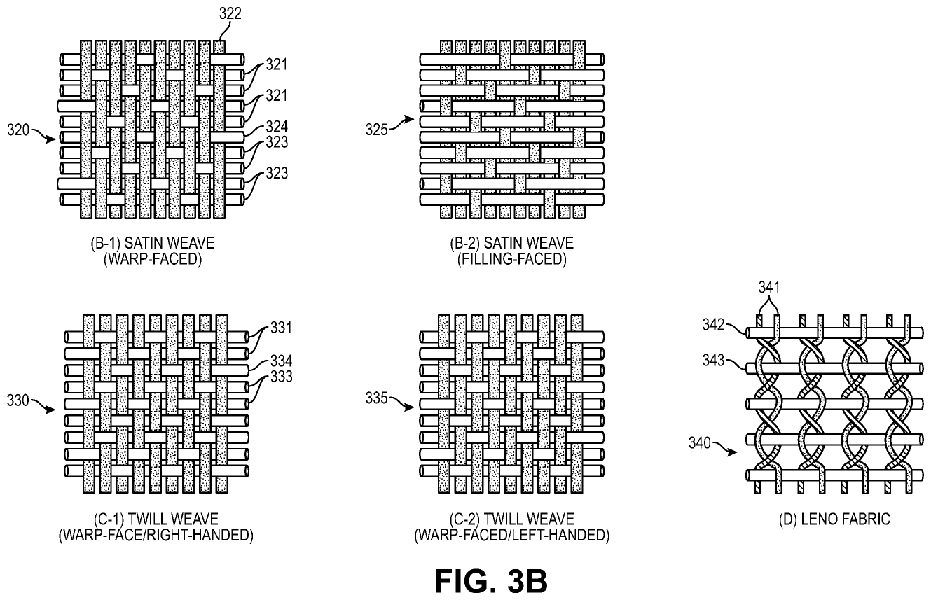

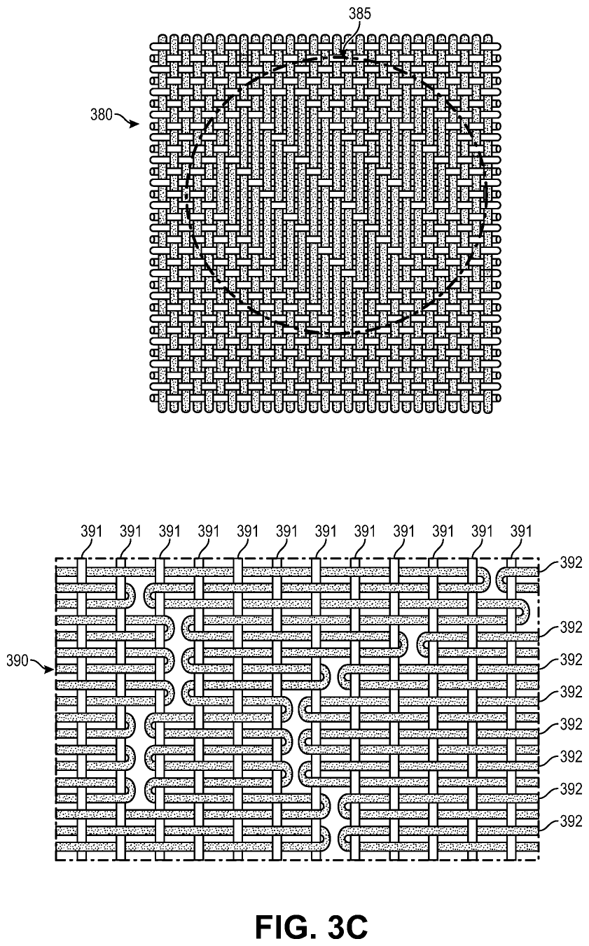

The example of FIG. 2 shows the warp threads 210 woven together with the weft threads 220 according to a plain weave. However, the warp threads 210 may be woven together with the weft threads 220 in any suitable configuration, and/or according to any suitable technique. In this regard, FIGS. 3A-3B show the warp threads 210 woven together with the weft threads 220 according to various configurations. For instance, FIG. 3A illustrates plain weave 305.

FIG. 3A further illustrates unbalanced plain weave 310. In some embodiments, the unbalanced plain weave is similar to the plain weave, except that threads running in the same direction may be in different dimensions. Also illustrated is a basket plain weave 315. In basket plain weave 315, double (warp and weft) strands alternate, rather than single strands alternating as in the plain weave and unbalanced plain weave configurations.

In FIG. 3B, in satin weave 320, the threads are grouped together such that a group of threads 321 (e.g., weft threads) running in a first direction passes underneath a thread 322 (e.g., a warp thread) running in a second direction. The group of threads 321 is separated from another group of threads 323 by separator thread 324. It should be understood that although the example of satin weave 320 illustrates groups of threads (e.g., 321 and 323) with four threads, the groups in a satin weave configuration may contain any number of threads with three or more threads. FIG. 3B further illustrates satin weave (filling-faced) 325.

FIG. 3B further illustrates twill weave 330. The twill weave 330 is similar to the satin weave 320, except that the groups are of two threads (rather than three or more threads). For example, the groups 331, 333 each comprise two threads, and a separated by separator thread 334. FIG. 3B further illustrates twill weave (warp-faced/left-handed) 335.

FIG. 3B further illustrates leno fabric weave 340. In the example leno fabric weave 340, the warp threads 341 pass on opposite sides of the weft threads 342. Further in the illustrated example of a leno fabric weave 340, the warp threads 341 alternate sides between weft threads. For example, if a warp thread 341 is on a right side of weft thread 343, it will be on a left side of weft thread 342. Furthermore, it should be understood that the leno fabric weave 340 is an illustrative example, and the leno fabric weave may be applied equally in either the warp or weft directions. For instance, a leno fabric weave may have weft threads pass on opposite sides of the warp threads, and so forth, analogously to the example of 340.

FIG. 3C illustrates two additional weaves, a Jacquard weave 380, and a Kesi weave 390. The Jacquard weave 380 is similar to a plain weave, except that some of the threads skip loops with other threads to form non-uniform pattern section 385. In the Kesi weave 390, the warp threads 391 simply flow vertically while the weft threads 392 loop around particular weft threads 391 to form a desired pattern. As will be seen, in some embodiments, the Jacquard weave 380 and/or Kesi weave 390 may be used to deliver electrical, optical, and/or chemical signals to targeted areas of the subject 120.

### Example Techniques

As mentioned above, the systems and methods described herein advantageously provide an implantable device 110 that is both: (i) multifunctional, and (ii) flexible yet structurally stable.

The multifunctionality, in some embodiments, is achieved by using different types of threads. The different types of threads may include electrical wire, permeable tubing, optical fiber, polymer fiber, surface functionalized fiber, absorbable suture, and/or fiber comprising material configured for sensing. In some embodiments, the woven portion 105 comprises only two different types of threads. However, in other embodiments, the woven portion may include any number of different types of threads.

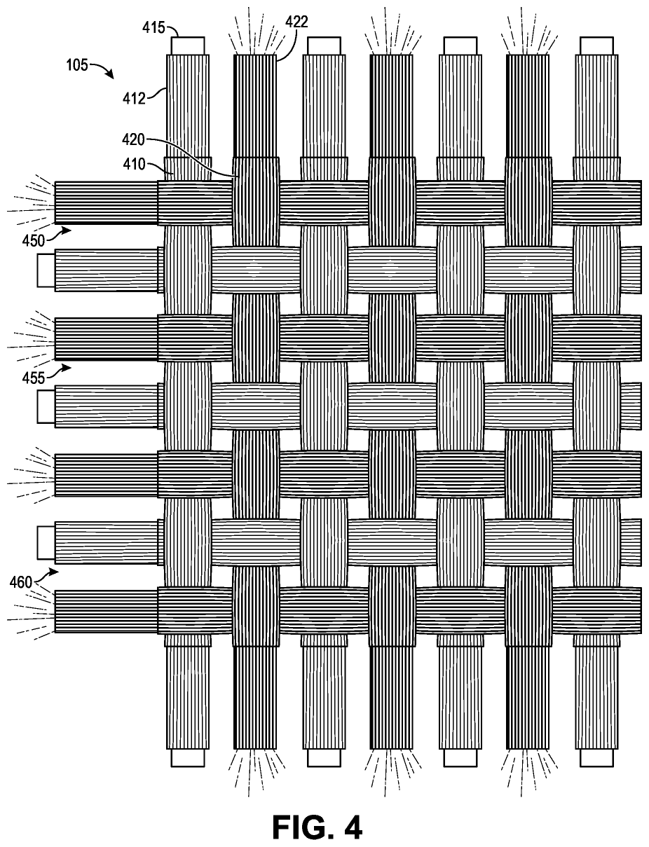

Electrical wire is used as a thread type in some embodiments. In this regard, FIG. 4 illustrates an example woven portion 105 including electrical wires 410. The electrical wires 410 may be of any suitable material. For example, the electrical wires 410 may comprise platinum, tungsten, and/or copper. Furthermore, in some embodiments, the electrical wires 410 may be insulated. In some implementations, the electrical wires 410 are 10 microns wide.

The electrical wires 410 may be used for any suitable purpose. For example, the electrical wires 410 may be used to deliver electrical stimulation, and/or measure electrical signals. To this end, the electrical wires 410 may be connected to electrodes 415. In some embodiments, a portion of the electrical wires 410 protrudes away (e.g., through a protrusion portion 412 of electrical wires 410) from a body of the woven portion 105 before the electrical wires 410 are connected to the electrodes 415. However, in other embodiments, the electrical wires 410 may end at edges of the woven portion 105 so that there is no protrusion portion 412 of the electrical wires 410; and, in some of these embodiments, the electrodes 415 are connected to the electrical wires 410 at the edges of the woven portion 105.

Any of the electrodes 415 may be configured to: deliver electrical stimulation (e.g., be a stimulation electrode), measure an electrical signal (e.g., be a sensing electrode), or both deliver electrical stimulation and measure an electrical signal. Any patterns for electrical stimulation and/or electrical measurement may be used. For example, in some embodiments, electrical stimulation is delivered to the subject 120 in a middle portion of the woven portion 105; and subsequently, electrical measurements are taken on end portions of the woven portion 105 to determine the subject's 120 reaction to the electrical stimulation.

In some embodiments, the electrical stimulation may be delivered from more specific locations of the woven portion 105. For example, rather than protrude from end portions of the woven portion 105 (as in the example of FIG. 4), the electrodes 415 may protrude from an interior of the woven portion 105, thereby delivering more targeted delivery of the electrical stimulation.

In some embodiments, the electrical interface 106 and/or the computing device 130 may be used wholly or partially to control delivery of the electrical stimulation and/or sensing of the electrical signal(s).

Optical fiber is used as a thread type in some embodiments. In this regard, the example of FIG. 4 also illustrates optical fibers 420. In the illustrated example, the optical fibers 420 protrude away (e.g., through protrusion portion 422) from the woven portion 105. However, it should be understood that in some embodiments there is no protrusion portion 422, and the optical fibers simply end at the edges of the woven portion 105.

The optical fibers 420 may be used for any suitable purpose. For example, the optical fibers 420 may be used to deliver optical stimulation, and/or measure optical signals. If optical stimulation is delivered, any suitable wavelength may be used for the optical stimulation. For instance, light with a wavelength between 500 and 600 nanometers may be used. The optical stimulation may be delivered from the optical instrument 109, which may be a fiber optic illuminator, a laser generator, etc.

In some embodiments, an optical stimulation pattern is controlled based on signals received from the electrical wires 410. For example, the computing device 130 may determine, based on signals received from the electrical wires 410 that a triggering condition has been met; then, in response to the triggering condition being met, control the optical fibers 420 to deliver optical stimulation to the subject 120. In some embodiments, there are multiple trigger conditions, and different optical stimulation patterns are delivered to the subject 120 depending on which triggering condition(s) is/are met.

In some implementations, the one or more of the optical fibers 420 is/are configured to measure signals.

In some embodiments, rather than protrude from end portions of the woven portion 105 (as in the example of FIG. 4), the optical fibers 420 may protrude from an interior of the woven portion 105, thereby delivering more targeted delivery or measurement of the optical signal(s).

In some embodiments, the electrical interface 106 and/or the computing device 130 may be used wholly or partially to control delivery of the optical stimulation and/or sensing of the optical signal(s). In this regard, a closed loop system may be created. For example, the state of the subject's 120 neural activity (e.g., as recorded from the electrical wires 410) may determine an optical stimulation pattern that is continuously updated. In some embodiments, the optical stimulation pattern is determined by the optical stimulation pattern generator 142.

Furthermore, it should be understood that the delivered optical stimulation advantageously allows for control of the subject's 120 neurons (e.g., through optogenetics, fluorescence stimulation, etc.).

Furthermore, it should be understood that any features of the electrical stimulation patterns and the optical stimulation patters may be controlled. For example, the patterns may be controlled in terms of time, space, intensity, etc. For example, regarding time, the patterns may be controlled to start and end at particular times. In an example regarding space, the pattern(s) may be controlled to deliver stimulation from the electrical wires 410 and/or the optical fibers 420 protruding from any of the upper portion 450 of the woven portion 105; the middle portion 455 of the woven portion 105; and/or the lower portion 460 of the woven portion 105. In some embodiments, the optical stimulation pattern may be calculated by the optical stimulation pattern generator 142. In some embodiments, the electrical stimulation pattern may be calculated by the electrical stimulation pattern generator 144.

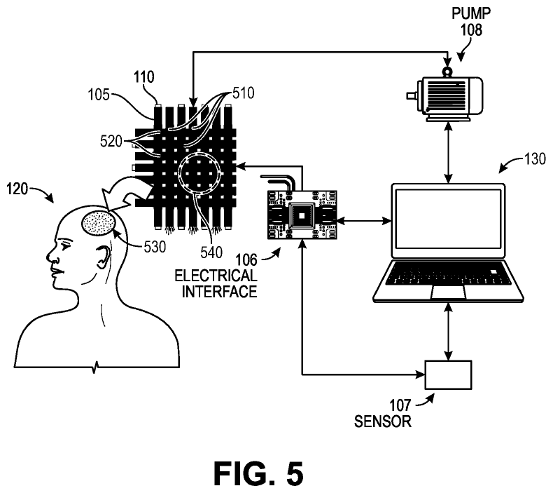

Permeable tubing is used as a thread type in some embodiments. In this regard, FIG. 5 illustrates an example of an implantable device 110, including permeable tubing 510.

The permeable tubing 510 may be used for any suitable purpose. For example, the permeable tubing 510 may be used to deliver a chemical or biological substance to the subject 120. In particular, the pump 108 may deliver the chemical or biological substance to the subject 120 through the permeable tubing 510. In some implementations, the pump 108 is a mechanical pump, and/or is controlled by the computing device 130.

In some embodiments, only a portion of the permeable tubing 510 is permeable, thus allowing targeted delivery of the chemical or biological substance. Put another way, the permeable tubing 510 may have a permeable portion (where it is desired to deliver the chemical or biological substance), and a non-permeable portion (where it is not desired to deliver the chemical or biological substance). It should be understood that this may allow targeted delivery of the chemical or biological substance from interior portion(s) and/or end portion(s) of the woven portion 105.

Any substance may be delivered through the permeable tubing 510 (e.g., to the lesion 530, or any other part of the subject 120). For example, the permeable tubing 510 may deliver a growth factor, such as brain derived neurotrophic factor (BDNF), or vascular endothelial growth factor (VEGF). In another example, the permeable tubing 510 may deliver an anti-inflammatory drug (e.g., Cyclosporin A, interleukin-1 receptor antagonist (IL-1Ra), etc.) to the subject 120. Moreover, the permeable tubing 510 may provide for targeted drug delivery, nutrition supply for cell culturing, oxygen supply for cell culturing, etc. Additional examples of the substance delivered through the permeable tubing 510 may include viral vectors (e.g., Adeno-associated virus vectors (AAVs), etc.), and mRNA.

In addition, the permeable tubing 510 may be used to bring a chemical or biological substance to the sensor 107 for sensing. The sensor 107 may be any type of sensor, and may sense any type of substance (e.g., to determine the type of the substance, a property of the substance, etc.). In one example, the sensor 107 may (based on the substance received from the permeable tubing 510) determine a blood oxygen level, a carbon dioxide level, a vascularization level, and/or a pH level of the substance.

Polymer fiber, is used as a thread type in some embodiments. In some embodiments, the polymer fiber serves as a structural thread. That is, the polymer fiber acts to provide structural support. For example, a structural thread (e.g., a polymer fiber, and/or absorbable suture) may provide better structural support than a functional thread (e.g., electrical wire, permeable tubing, optical fiber, surface functionalized fiber, and/or fiber comprising material configured for sensing). In this regard, in some embodiments, one or more structural threads are woven together with one or more functional threads, thus providing for structure and functionality. For instance, in the example of FIG. 5, the polymer fibers 520 are woven together with the permeable tubing 510 so that the polymer fibers 520 provide structural support to the permeable tubing 510.

The polymer fiber may be made of any suitable material. For example, the polymer fiber may be made of polypropylene, polyvinyl chloride (PVC), polyethylene, polyether ether ketone (PEEK), polycarbonate, polyetherimide (PEI), polysulfone, polyurethane, cellulose, and/or chitosan.

Surface functionalized fiber, is used as a thread type in some embodiments. In some implementations, the surface functionalized fiber is used to modify a surface binding of the woven portion 105. Additionally or alternatively, the surface functionalized fiber may be used for chemical elution. For example, chemicals (e.g., antibodies, etc.) may be attached to the fibers; and, these chemicals may provide functions such as stimulation, sensing and selection with specific spatial distributions (the spatial distributions depending, e.g., on how the chemicals are attached and/or how the threads are woven).

Examples of materials that the surface functionalized fiber may be made of include polypropylene, polyacrylonitrile, carbon fiber, cellulose, and/or cotton.

Absorbable suture, is used as a thread type in some embodiments. In some implementations, the absorbable suture serves as a structural thread. That is, the absorbable suture acts to provide structural support. However, in contrast to the polymer fiber, in some embodiments, the absorbable suture is biodegradable. Thus, the structural support provided by the absorbable suture may be temporary. Example materials that the absorbable suture may be made of include catgut, collagen, aliphatic polyesters, polyglycolic acid, poly(glycolide-co-L-lactide) copolymer (PGA), poly-p-dioxanone (PDS), polylactide (PLA), and polyhydroxyalkanoates (PHA).

Fiber comprising material configured for sensing is used as a thread type in some embodiments. The fiber comprising material configured for sensing may comprise any suitable type of material, and sense any parameter. For example, the material may comprise a piezoelectric material, and be configured to sense a heart rate of the subject 120. In this example, it should be understood that the sensor 107 may be used to measure changes in pressure (and thereby measure the heartbeat of the subject 120) based on the piezoelectric effect. Additionally or alternatively, in embodiments where the fiber comprising material configured for sensing is a piezoelectric material, the sensor 107 may measure temperature, acceleration, strain, force, etc.

### Example Methods

As mentioned above, the implantable device 110 may be used, inter alia, to repair a lesion.

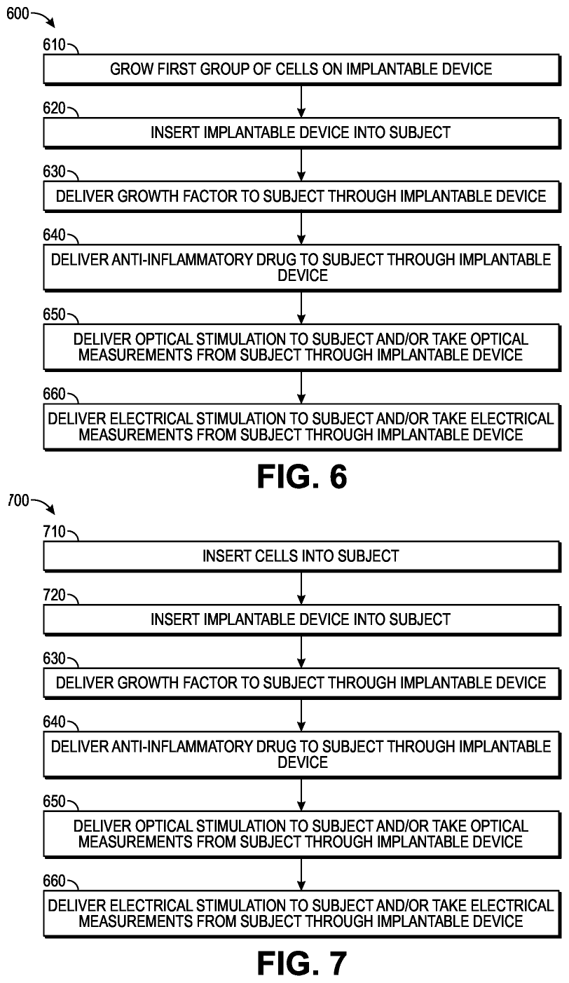

In this regard, FIG. 6 illustrates an example method 600 of repairing a lesion 530, including growing a first group of cells 540 on the implantable device 110 prior to insertion of the implantable device 110 into the subject 120. It should be understood that the method 600 is only an example; and the method 600 may include additional, fewer, or alternate actions, including those discussed elsewhere herein.

The example method 600 begins at block 610 when a first group of cells 540 is grown on the implantable device 110. In some embodiments, the cells 540 may be grown for the purpose of repairing a lesion 530 (e.g., a tumor, etc.). The cells 540 grown on the implantable device 110 may include any type of cells. For example, the cells 540 may include induced pluripotent stem cells (iPSCs), neural stem cells (NSCs), and/or neurons.

In some embodiments, the cells 540 are grown (e.g., in vitro, etc.) on a structural thread (e.g., polymer fiber or absorbable suture), thus providing for greater stability. In embodiments where the cells 540 are grown on the absorbable suture, the absorbable suture may biodegrade thus allowing the cells 540 to flow more freely into the lesion 530.

At block 620, the implantable device 110 may be inserted into the subject 120. For example, the implantable device may be inserted into the lesion 540. In this regard, it may be noted that, in some embodiments, the implantable device 110 both aids in the repair of the lesion 540, and monitors the progress of the subject 120. This is advantageous over prior systems for lesion repair because prior systems for lesion repair lack an effective way to monitor progress of the subject 120.

At block 630, a growth factor may be delivered to the subject 120 (e.g., through the permeable tubing 510 and/or pump 108). The growth factor may be for any purpose, such as stimulating cell proliferation (e.g., of the group of cells 540), and/or wound healing. Any type of growth factor may be used. For example, the growth factor may be derived neurotrophic factor (BDNF) and/or vascular endothelial growth factor (VEGF).

In some embodiments, the growth factor is inserted into the subject following a predetermined time period (e.g., 1 day, 2 days, etc.) after insertion of the implantable device 110 into the subject 120. This allows the first group of cells 540 to exist in the subject for the predetermined time period before being exposed to the growth factor. The length of the predetermined time period may be determined based on any suitable criteria, such as type of growth factor, amount of growth factor, type of lesion, size of lesion, location of lesion, characteristics of the subject 120 (e.g., age, weight, gender, underlying health/risk conditions, etc.), etc.

Additionally or alternatively, the growth factor may be inserted into the subject based upon a predetermined criteria being met. For example, when a predetermined level of vascularization in the lesion 530 (e.g., as measured via the permeable tubing 510 and sensor 107), the growth factor may be delivered.

In some embodiments, the growth factors are inserted (e.g., via the permeable tubing 510) into the tissue surrounding the lesion 530 to improve the survival and integration of cells 540.

At block 640, an anti-inflammatory drug may be delivered to the subject 120 (e.g., through the permeable tubing 510 and/or pump 108). The anti-inflammatory drug may, for example, reduce inflammation in or around the lesion 530, and/or promote wound healing. Examples of the anti-inflammatory drug include Cyclosporin A, interleukin-1 receptor antagonist (IL-1Ra), etc.

In some embodiments, the anti-inflammatory drug is inserted into the subject following a predetermined time period (e.g., 1 day, 2 days, etc.) after insertion of the implantable device 110 into the subject 120. This allows the first group of cells 540 to exist in the subject for the predetermined time period before being exposed to the anti-inflammatory drug. The length of the predetermined time period may be determined based on any suitable criteria, such as type of anti-inflammatory drug, amount of anti-inflammatory drug, type of lesion, size of lesion, location of lesion, characteristics of the subject 120 (e.g., age, weight, gender, underlying health/risk conditions, etc.), etc.

Additionally or alternatively, the anti-inflammatory drug may be inserted into the subject based upon a predetermined criteria being met. For example, when a predetermined level of vascularization in the lesion 530 (e.g., as measured via the permeable tubing 510 and sensor 107), the anti-inflammatory drug may be delivered.

At block 650, optical stimulation may be delivered to the subject 120 (e.g., via the optical fiber 420, and/or optical instrument 109). The delivered optical stimulation may promote healing of the subject 120, particularly in an area of the lesion 530. Furthermore, as described above, the optical stimulation may be delivered as part of a closed loop system. For instance, the optical stimulation pattern may be determined (e.g., by the optical stimulation pattern generator 142) in a closed loop system with inputs of, for example, the optical measurements taken via the implantable device 110, electrical measurements taken via the implantable device 110 (e.g., at block 660), etc.

At block 660, electrical stimulation may be delivered to the subject 120 (e.g., via the electrical wires 410, and/or electrical interface 106). The delivered electrical stimulation may be delivered to any area of the subject 120, including wholly or partially into the lesion 530. Furthermore, the electrical stimulation may be delivered as part of a closed loop system. For instance, the electrical stimulation pattern may be determined (e.g., by the electrical stimulation pattern generator 144) in a closed loop system with inputs of, for example, the optical measurements taken via the implantable device 110 (e.g., at block 650), electrical measurements taken via the implantable device 110 (e.g., at block 660), etc.

Furthermore, regarding blocks 630, 640, 650, 660, it should be understood that the subject may periodically return to a treatment center (e.g., a hospital, a doctor's office, a medical facility, a laboratory, etc.) to have one or more of these blocks administered. Advantageously, in some embodiments, the implantable device need not be removed between visits. The amount of visits to the treatment center may be subject 120 specific. For instance, if a treatment plan for a particular subject 120 was to receive optical stimulation every other day, the particular subject 120 would return to the treatment center every other day.

FIG. 7 illustrates an example method 700 of repairing a lesion 530, including inserting a first group of cells into the subject 120 prior to inserting the implantable device 110 into the subject 120. It should be understood that the method 700 is only an example; and the method 700 may include additional, fewer, or alternate actions, including those discussed elsewhere herein.

The example method 700 begins at block 710 when a first group of cells 540 is inserted into the subject 120. In some embodiments, the cells 540 may be inserted with a syringe and needle. The inserted cells may include any type of cells. For example, the cells may include iPSCs, NSCs, and/or neurons. The cells may be grown in vitro prior to insertion to the subject 120.

In some embodiments, the cells are inserted into the lesion 530 for the purpose of repairing the lesion 530.

At block 720, the implantable device 110 may be inserted into the subject 120. The implantable device may be inserted immediately after the insertion of the cells at block 710. Alternatively, the implantable device 110 may be inserted following a predetermined time period after the insertion of the cells at block 710. The length of the predetermined time period may be determined based on any suitable criteria, such as type of cells, treatment to be administered (e.g., if a growth factor will be administered, etc.), type of lesion, size of lesion, location of lesion, characteristics of the subject 120 (e.g., age, weight, gender, underlying health/risk conditions, etc.), etc.

In the example method 700, blocks 630, 640, 650, and 660 may be performed substantially similarly to blocks 630, 640, 650, and 660 in the example method 600 of FIG. 6.

Further regarding the example flowcharts provided above, it should be noted that all blocks are not necessarily required to be performed. Moreover, additional blocks may be performed although they are not specifically illustrated in the example flowcharts. In addition, the example flowcharts are not mutually exclusive. For example, block(s) from one example flowchart may be performed in another of the example flowcharts.

### Additional Exemplary Embodiments

Aspect 1. An implantable device, comprising:
a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and
a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing.
Aspect 2. The implantable device of aspect 1, further comprising a third plurality of threads, the third plurality of threads orientated in the first direction, the third plurality of threads being woven together with the second plurality of threads, the third plurality of threads comprising a thread type that is different from both the first thread type and the second thread type, the third thread type comprising: electrical wire, permeable tubing, polymer fiber, optical fiber, surface functionalized fiber, absorbable suture, or fiber comprising material configured for sensing.
Aspect 3. The implantable device of aspect 2, further comprising a fourth plurality of threads, the fourth plurality of threads orientated in the second direction, the fourth plurality of threads being woven together with the first plurality of threads and the third plurality of threads, the fourth plurality of threads comprising a fourth thread type that is different from all of the first, second, and third thread types, and the fourth thread type comprising: electrical wire, permeable tubing, optical fiber, polymer fiber, surface functionalized fiber, absorbable suture, or fiber comprising material configured for sensing.
Aspect 4. The implantable device of aspect 3, further comprising a fifth plurality of threads, the fifth plurality of threads orientated in the first direction, the fifth plurality of threads being woven together with the second and fourth plurality of threads, the fifth plurality of threads comprising a fifth thread type that is different from all of the first, second, third, and fourth thread types, the and the fourth thread type comprising: electrical wire, permeable tubing, optical fiber, polymer fiber, surface functionalized fiber, absorbable suture, or fiber comprising material configured for sensing.
Aspect 5. The implantable device of any one of aspects 1-4, the first thread type comprising the electrical wire, at least one electrical wire of the first plurality of threads comprising an electrode connected to an end of the at least one electrical wire.
Aspect 6. The implantable device of one of aspects 1-5, the first thread type comprising the electrical wire, at least one electrical wire of the first plurality of threads configured to protrude away from a woven portion of the implantable device.
Aspect 7. The implantable device of one of aspects 1-6, the first thread type comprising the permeable tubing, and the device further comprising a pump: (i) connected to the first plurality of threads, and (ii) configured to drive a chemical or biological substance through at least one permeable tube of the first plurality of threads.
Aspect 8. The implantable device of one of aspects 1-7, the thread type comprising the permeable tubing, and the device further comprising a sensor configured to sense a chemical or biological substance received through at least one permeable tube of the first plurality of threads.
Aspect 9. The implantable device of one of aspects 1-8, the first thread type comprising the permeable tubing, and the device further comprising a sensor configured to: (i) receive a substance from at least one thread of the first plurality of threads, and (ii) sense, based on the substance, at least one of: a blood oxygen level, a carbon dioxide level or a pH level.
Aspect 10. The implantable device of one of aspects 1-9, the first thread type comprising the optical fiber, at least one optical fiber of the first plurality of threads configured to protrude away from a woven portion of the implantable device.
Aspect 11. The implantable device of one of aspects 1-10, the first thread type comprising the optical fiber, the implantable device further comprised in a system including an optical instrument configured to deliver optical stimulation through at least one optical fiber of the first plurality of threads, the optical stimulation comprising light with a wavelength between 500 and 600 nanometers.
Aspect 12. The implantable device of one of aspects 1-11,
the first thread type comprising the electrical wire, and the second thread type comprising the optical fiber; and
the implantable device further comprised in a system, the system comprising:
   an electrical interface: (i) connected to at least one electrical wire of the first plurality of threads, and (ii) configured to deliver electrical stimulation through the at least one electrical wire; and
   an optical instrument: (i) connected to at least one optical fiber of the second plurality of threads, and (ii) configured to deliver optical stimulation through the at least one optical fiber.
Aspect 13. The implantable device of one of aspects 1-12,
the first thread type comprising the electrical wire, and the second thread type comprising the optical fiber; and
the implantable device further comprised in a system, the system comprising:
   an electrical interface: (i) connected to at least one electrical wire of the first plurality of threads, and (ii) configured to receive an electrical signal from the at least one electrical wire;
   an optical instrument connected to at least one optical fiber of the second plurality of thread; and
   one or more processors configured to: (i) receive the electrical signal from the electrical interface, (ii) determine an optical stimulation pattern based on the received electrical signal, and (iii) control the optical instrument to deliver the determined optical stimulation pattern through the at least one optical fiber.
Aspect 14. The implantable device of one of aspects 1-13,
the first thread type comprising the electrical wire, and the second thread type comprising the optical fiber; and
the implantable device further comprising a third plurality of threads, the third plurality of threads orientated in the first or second directions, the third plurality of threads being woven together with the first or second plurality of threads, the third plurality of threads comprising a thread type comprising permeable tubing.
Aspect 15. The implantable device of aspect 14,
the first second and third pluralities of threads comprised in a woven portion of the device;
the woven portion of the device comprising: (i) an upper portion, (ii) a middle portion, (iii) a lower portion; and
at least one end of an optical fiber of the second plurality of threads is configured to protrude: (i) away from the upper portion or lower portion, and (ii) into a subject.
Aspect 16. The implantable device of one of aspects 1-15, the first thread type comprising the polymer fiber.
Aspect 17. The implantable device of one of aspects 1-16, the first thread type comprising the surface functionalized fiber.
Aspect 18. The implantable device of one of aspects 1-17, the first thread type comprising the absorbable suture, the absorbable suture comprised of a biodegradable material.
Aspect 19. The implantable device of one of aspects 1-18,
the first thread type comprising the fiber comprising the material configured for sensing, the material configured for sensing comprising a piezoelectric material; and
the implantable device further comprising a sensor configured to measure a heart rate of a patient by measuring a change in resistance of at least one thread of the first plurality of threads comprising the piezoelectric material.
Aspect 20. The implantable device of one of aspects 1-19,
the first thread type comprising the fiber comprising the material configured for sensing; and
the material configured for sensing being configured to sense a temperature.
Aspect 21. The implantable device of one of aspects 1-20,
the first thread type comprising: polymer fiber, surface functionalized fiber, absorbable suture, or fiber comprising material configured for sensing; and
the second thread type comprising: polymer fiber, surface functionalized fiber, absorbable suture, or fiber comprising material configured for sensing.
Aspect 22. The implantable device of one of aspects 1-21, the first plurality of threads and the second plurality of threads each having a diameter of between 1-100 μM.
Aspect 23. The implantable device of one of aspects 1-22, wherein the first plurality of threads is woven together with the second plurality of threads according to:
a plain weave;
a basket weave;
a satin weave;
a twill weave; or
a leno fabric weave.
Aspect 24. The implantable device of one of aspects 1-23,
the first plurality of threads comprising a plurality of structural threads, the first thread type comprising either polymer fiber or absorbable suture; and
the second plurality of threads comprising a functional thread, the second thread type comprising electrical wire, permeable tubing, optical fiber, surface functionalized fiber, or fiber comprising material configured for sensing.
Aspect 24. A method of repairing a lesion, the method comprising:
growing a first group of cells on an implantable device, the first group of cells comprising induced pluripotent stem cells (iPSCs), neural stem cells (NSCs), or neurons, and the implantable device comprising:
   (i) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and
   (ii) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing; and
inserting the implantable device into a subject.
Aspect 25. The method of aspect 24, the first thread type comprising the permeable tubing, and the method further comprising:
delivering a growth factor to the subject through at least one permeable tube of the first plurality of threads.
Aspect 26. The method of aspect 25, the growth factor being brain derived neurotrophic factor (BDNF) or vascular endothelial growth factor (VEGF).
Aspect 27. The method of any one of aspects 24-25, the delivering of the growth factor to the subject occurring after a predetermined time period of one day following the inserting of the implantable device into the subject.
Aspect 28. The method of any one of aspects 23-27, the first thread type comprising the permeable tubing, and the method further comprising:
delivering an anti-inflammatory drug to the subject through at least one permeable tube of the first plurality of threads.
Aspect 29. The method of any one of aspects 23-28, the first thread type comprising the optical fiber, and the method further comprising:
delivering optical stimulation to the subject though at least one optical fiber of the first plurality of threads.
Aspect 30. The method of any one of aspects 23-29, the second thread type comprising the electrical wire, and the method further comprising:
delivering electrical stimulation to the subject though at least one electrical wire of the second plurality of threads.
Aspect 31. The method of any one of aspects 23-30, the inserting the implantable device into the subject comprising inserting the implantable device into the lesion.
Aspect 32. The method of any one of aspects 23-31, the inserting the implantable device into the subject comprising inserting the implantable device into a tumor of the subject.
Aspect 33. A method of repairing a lesion, the method comprising:
inserting a first group of cells into the subject, the first group of cells comprising induced pluripotent stem cells (iPSCs), neural stem cells (NSCs), or neurons; and
inserting an implantable device into a subject, the implantable device comprising:
   (i) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and
   (ii) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing.
Aspect 34. The method of aspect 33, the first thread type comprising the permeable tubing, and the method further comprising:
delivering a growth factor to the subject through at least one permeable tube of the first plurality of threads.
Aspect 35. The method of aspect 34, the growth factor being brain derived neurotrophic factor (BDNF) or vascular endothelial growth factor (VEGF).
Aspect 36. The method of any one of aspects 33-35, the first thread type comprising the permeable tubing, and the method further comprising:
delivering an anti-inflammatory drug to the subject through at least one permeable tube of the first plurality of threads.
Aspect 37. The method of any one of aspects 33-36, the first thread type comprising the optical fiber, and the method further comprising:
delivering optical stimulation to the subject though at least one optical fiber of the first plurality of threads.
Aspect 38. The method of any one of aspects 33-37, the second thread type comprising the electrical wire, and the method further comprising:
delivering electrical stimulation to the subject though at least one electrical wire of the second plurality of threads.
Aspect 40. The method of any one of aspects 33-39,
the first plurality of threads comprising a plurality of structural threads, the first thread type comprising either polymer fiber or absorbable suture; and
the second plurality of threads comprising a functional thread, the second thread type comprising electrical wire, permeable tubing, optical fiber, surface functionalized fiber, or fiber comprising material configured for sensing.

### Other Matters

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Furthermore, the patent claims at the end of this patent application are not intended to be construed under 35 U.S.C. § 112 (f) unless traditional means-plus-function language is expressly recited, such as “means for” or “step for” language being explicitly recited in the claim(s). The systems and methods described herein are directed to an improvement to computer functionality, and improve the functioning of conventional computers.

1. An implantable device, comprising:
a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and
a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing.

2. The implantable device of claim 1, further comprising a third plurality of threads, the third plurality of threads orientated in the first direction, the third plurality of threads being woven together with the second plurality of threads, the third plurality of threads comprising a thread type that is different from both the first thread type and the second thread type, the third thread type comprising: electrical wire, permeable tubing, polymer fiber, optical fiber, surface functionalized fiber, absorbable suture, or fiber comprising material configured for sensing.

3. The implantable device of claim 2, further comprising a fourth plurality of threads, the fourth plurality of threads orientated in the second direction, the fourth plurality of threads being woven together with the first plurality of threads and the third plurality of threads, the fourth plurality of threads comprising a fourth thread type that is different from all of the first, second, and third thread types, and the fourth thread type comprising: electrical wire, permeable tubing, optical fiber, polymer fiber, surface functionalized fiber, absorbable suture, or fiber comprising material configured for sensing.

4. The implantable device of claim 3, further comprising a fifth plurality of threads, the fifth plurality of threads orientated in the first direction, the fifth plurality of threads being woven together with the second and fourth plurality of threads, the fifth plurality of threads comprising a fifth thread type that is different from all of the first, second, third, and fourth thread types, the and the fourth thread type comprising: electrical wire, permeable tubing, optical fiber, polymer fiber, surface functionalized fiber, absorbable suture, or fiber comprising material configured for sensing.

5. The implantable device of claim 1, the first thread type comprising the electrical wire, at least one electrical wire of the first plurality of threads comprising an electrode connected to an end of the at least one electrical wire.

6. The implantable device of claim 1, the first thread type comprising the electrical wire, at least one electrical wire of the first plurality of threads configured to protrude away from a woven portion of the implantable device.

7. The implantable device of claim 1, the first thread type comprising the permeable tubing, and the device further comprising a pump: (i) connected to the first plurality of threads, and (ii) configured to drive a chemical or biological substance through at least one permeable tube of the first plurality of threads.

8. The implantable device of claim 1, the thread type comprising the permeable tubing, and the device further comprising a sensor configured to sense a chemical or biological substance received through at least one permeable tube of the first plurality of threads.

9. The implantable device of claim 1, the first thread type comprising the permeable tubing, and the device further comprising a sensor configured to: (i) receive a substance from at least one thread of the first plurality of threads, and (ii) sense, based on the substance, at least one of: a blood oxygen level, a carbon dioxide level or a pH level.

10. The implantable device of claim 1, the first thread type comprising the optical fiber, at least one optical fiber of the first plurality of threads configured to protrude away from a woven portion of the implantable device.

11. The implantable device of claim 1, the first thread type comprising the optical fiber, the implantable device further comprised in a system including an optical instrument configured to deliver optical stimulation through at least one optical fiber of the first plurality of threads, the optical stimulation comprising light with a wavelength between 500 and 600 nanometers.

12. The implantable device of claim 1,
the first thread type comprising the electrical wire, and the second thread type comprising the optical fiber; and
the implantable device further comprised in a system, the system comprising:
an electrical interface: (i) connected to at least one electrical wire of the first plurality of threads, and (ii) configured to deliver electrical stimulation through the at least one electrical wire; and
an optical instrument: (i) connected to at least one optical fiber of the second plurality of threads, and (ii) configured to deliver optical stimulation through the at least one optical fiber.

13. The implantable device of claim 1,
the first thread type comprising the electrical wire, and the second thread type comprising the optical fiber; and
the implantable device further comprised in a system, the system comprising:
an electrical interface: (i) connected to at least one electrical wire of the first plurality of threads, and (ii) configured to receive an electrical signal from the at least one electrical wire;
an optical instrument connected to at least one optical fiber of the second plurality of thread; and
one or more processors configured to: (i) receive the electrical signal from the electrical interface, (ii) determine an optical stimulation pattern based on the received electrical signal, and (iii) control the optical instrument to deliver the determined optical stimulation pattern through the at least one optical fiber.

14. The implantable device of claim 1,
the first thread type comprising the electrical wire, and the second thread type comprising the optical fiber; and
the implantable device further comprising a third plurality of threads, the third plurality of threads orientated in the first or second directions, the third plurality of threads being woven together with the first or second plurality of threads, the third plurality of threads comprising a thread type comprising permeable tubing.

15. The implantable device of claim 14,
the first second and third pluralities of threads comprised in a woven portion of the device;
the woven portion of the device comprising: (i) an upper portion, (ii) a middle portion, (iii) a lower portion; and
at least one end of an optical fiber of the second plurality of threads is configured to protrude: (i) away from the upper portion or lower portion, and (ii) into a subject.

16-24. (canceled)

25. A method of repairing a lesion, the method comprising:
growing a first group of cells on an implantable device, the first group of cells comprising induced pluripotent stem cells (iPSCs), neural stem cells (NSCs), or neurons, and the implantable device comprising:
(i) a first plurality of threads, the first plurality of threads orientated in a first direction, the first plurality of threads comprising a first thread type that is: electrical wire, permeable tubing, optical fiber, polymer fiber, surface functionalized fiber, absorbable suture, or fiber comprising material configured for sensing; and
(ii) a second plurality of threads, the second plurality of threads orientated in a second direction perpendicular to the first direction, the second plurality of threads being woven together with the first plurality of threads, the second plurality of threads comprising a second thread type that is different than the first thread type, the second thread type comprising: electrical wire, permeable tubing, optical fiber, polymer fiber, surface functionalized fiber, absorbable suture, or fiber comprising material configured for sensing; and
inserting the implantable device into a subject.

26. The method of claim 25, the first thread type comprising the permeable tubing, and the method further comprising:
delivering a growth factor to the subject through at least one permeable tube of the first plurality of threads.

27. The method of claim 26, the growth factor being brain derived neurotrophic factor (BDNF) or vascular endothelial growth factor (VEGF).

28. The method of claim 26, the delivering of the growth factor to the subject occurring after a predetermined time period of one day following the inserting of the implantable device into the subject.

29. The method of claim 25, the first thread type comprising the permeable tubing, and the method further comprising:
delivering an anti-inflammatory drug to the subject through at least one permeable tube of the first plurality of threads.

30-40. (canceled)
